(19) United States
(12) Patent Application Publication
(10) Pub. No.: US 2026/0007328 A1
(43) Pub. Date: Jan. 8, 2026

(54) BREATH ANALYSIS DEVICES, SYSTEMS, AND METHODS FOR DETECTING BACTERIAL INFECTIONS

(71) Applicant: THE BOARD OF TRUSTEES OF THE LELAND STANFORD JUNIOR UNIVERSITY, Stanford, CA (US)

(72) Inventors: Juliana Perl, Stanford, CA (US); Christopher Strand, Stanford, CA (US); Janene H. Fuerch, Stanford, CA (US); James Wall, Stanford, CA (US); Brandon Felkins, San Francisco, CA (US)

(21) Appl. No.: 19/325,394

(22) Filed: Sep. 10, 2025

Related U.S. Application Data
(63) Continuation of application No. PCT/US24/29325, filed on May 14, 2024.
(60) Provisional application No. 63/452,694, filed on Mar. 16, 2023.

(51) Int. Cl. A61B 5/083; A61B 5/00; A61B 5/097

(52) U.S. Cl. CPC A61B 5/0836; A61B 5/097; A61B 5/742; A61B 2503/045

(57) ABSTRACT

A breath analysis device for analyzing breath from a subject to detect bacterial infection, such a necrotizing enterocolitis. In one example, the device is configured for capturing breaths substantially continuously from a subject over a period of multiple breath cycles and includes a housing including an inlet for delivering breath from a subject to gas sensors that measure gasses including hydrogen. A processor is coupled to the sensors to analyze signals from the sensors to measure quantities of the gasses in the breath and to present information related to the measured quantities of the gasses on a display.

### RELATED APPLICATION DATA

The present application is continuation of co-pending International Application No. PCT/US2024/029325, filed Mar. 16, 2024, which claims benefit of U.S. provisional application Ser. No. 63/452,694, filed Mar. 16, 2023, the entire disclosures of which are expressly incorporated by reference herein.

### STATEMENT REGARDING FEDERALLY SPONSORED RESEARCH AND DEVELOPMENT

None.

### TECHNICAL FIELD

The present application relates to devices for continuous breath collection and analysis of breath from a subject and, more particularly, to devices for analyzing breath to detect bacterial infections in a subject, such as necrotizing enterocolitis or sepsis, and to systems and methods for using such devices.

### BACKGROUND

Necrotizing enterocolitis (NEC) is a life-threatening illness almost exclusively affecting neonates. Late-stage NEC has a mortality rate as high as 50%. The pathophysiology of NEC is related to inflammation of the intestine and bacterial infection, which causes cellular damage and can lead to necrosis of the intestines. As NEC progresses, it can lead to intestinal perforation causing peritonitis, sepsis, and death. The signs and symptoms of NEC, i.e., poor feeding, vomiting, lethargy, abdominal tenderness, are nonspecific, so clinicians must remain suspicious when presented with these signs and symptoms in the neonatal population.

Necrotizing enterocolitis is caused by bacterial invasion into the intestinal wall. This leads to inflammation and cellular destruction of the wall of the intestine. If unrecognized and untreated, an intestinal perforation may occur, causing spillage of intestinal contents into the peritoneum and resulting in peritonitis. The specific mechanism and cause of this bacterial invasion are not yet understood. In premature neonates, gastrointestinal tract immaturity is believed to play a role in the pathogenesis of necrotizing enterocolitis.

Necrotizing enterocolitis commonly affects neonates and is the most common life-threatening emergency affecting the gastrointestinal tract of infants in the neonatal intensive care unit. Necrotizing enterocolitis typically occurs in the first month of life. Several risk factors have been identified, but prematurity, low birth weight, and formula feeding have been identified as primary risks. Specifically, high osmotic strength formula feeding has been implicated as a risk factor. Genetic factors may also play a role. Incidence worldwide varies between 0.3 to 2.4 infants per 1000 live births. Most of these cases occur in premature infants born before 36 weeks gestation. Necrotizing enterocolitis affects 2% to 10% of all premature infants. Overall, mortality ranges from 10% to 50%. However, in the most severe cases, involving perforation, peritonitis, and sepsis, mortality approaches 100%.

While necrotizing enterocolitis primarily occurs in premature infants, it has been documented in full-term infants. In this population, onset is typically in the first few days of life and is usually associated with a hypoxic event, such as a cyanotic congenital heart defect.

Importantly, once identified, NEC has an established treatment paradigm consisting of antibiotics and restrictions to parenteral nutrition. Therefore, early detection and treatment can lead to improved patient outcomes.

Accordingly, devices and methods that facilitate detecting and/or identifying necrotizing enterocolitis in neonates and other patients would be useful.

### SUMMARY

The present application is directed to devices for analyzing breath from a subject. More particularly, the present application is directed to devices for analyzing breath to detect bacterial infections in a subject, such as necrotizing enterocolitis or sepsis, and to systems and methods for using such devices.

In accordance with one example, a device is provided for analyzing breaths of a subject that includes a housing comprising an inlet for delivering exhalation air from a subject into the housing; a hydrogen sensor within the housing; a carbon dioxide sensor within the housing; a display; and a processor coupled to the hydrogen sensor, the carbon dioxide sensor, and the display. The processor may be configured to analyze signals from the carbon dioxide sensor to confirm that breath samples comprise exhalation from the subject; analyze signals from the hydrogen sensor to measure hydrogen in the confirmed breath samples; and present information related to the measured quantity of hydrogen in the breath samples on the display.

In accordance with another example, a device is provided for analyzing breaths of a subject that includes a housing comprising an inlet for delivering exhalation gas substantially continuously from a subject into the housing; a plurality of gas sensors within the housing configured to measure gases including one or more of hydrogen, methane, carbon dioxide, and hydrogen sulfide; a display; and a processor coupled to the sensors to analyze signals from the sensors to measure quantities of the gases in a series of breath samples of the exhalation gas delivered into the housing and coupled to the display for presenting information related to the measured quantities of the gases in the breath samples.

In accordance with one example, a device is provided for analyzing the breaths of a subject that includes an apparatus for the continuous capture of breaths over a period of multiple breath cycles; a housing comprising an inlet for delivering breath from a subject into the housing; a hydrogen sensor within the housing; a display; and a processor coupled to the hydrogen sensor to analyze signals and measure hydrogen gas in the breath and coupled to the display for presenting information related to the measured quantity of hydrogen in the breaths.

Optionally, the device may include one or more additional gas sensors and the processor may present information related to the quantities of one or more additional gasses, such as methane, carbon dioxide, and hydrogen sulfide. In addition or alternatively, the device may measure carbon dioxide or other parameters of the breath cycle(s) to confirm that the gasses being analyzed correspond to actual breaths from the subject.

In accordance with another example, a device is provided for analyzing breaths of a subject that includes an apparatus for substantially continuous capture of breath over a period of multiple breath cycles; a housing comprising an inlet for delivering breath from a subject into the housing; a plurality of gas sensors within the housing configured to measure gasses including one or more of hydrogen, methane, carbon dioxide, and hydrogen sulfide; a display; and a processor coupled to the sensors to analyze signals from the sensors to measure quantities of the gasses in the breaths and coupled to the display for presenting information related to the measured quantities of the gasses in the breaths.

In accordance with still another example, a system is provided for analyzing breaths from a subject to detect infection that includes a device for analyzing breath of a subject including an apparatus for substantially continuous capture of breaths over a period of multiple breath cycles; a housing comprising an inlet for delivering breaths from a subject into the housing; one or more gas sensors within the housing configured to measure gasses including one or more of hydrogen, methane, carbon dioxide, and hydrogen sulfide; a display; and a processor coupled to the sensors to analyze signals from the one or more sensors to measure quantities of the gasses in the breaths and coupled to the display for presenting information related to the measured quantities of the gasses in the breaths. For example, the apparatus may include one or more of tubing including a first end coupled to the inlet; a nasal cannula; a face mask; and medical equipment configured to receive exhalation breaths from the subject, such as a ventilator, capnography device, and the like, which may be coupled to the housing to deliver breath gasses from the subject to the device.

In accordance with yet another example, a method is provided for analyzing breath over a period of multiple breath cycles from a subject to detect infection that includes capturing breath over a period of multiple breath cycles, delivering breath from the subject into a breath analysis device to expose the breath to one or more gas sensors, whereupon a processor coupled to the sensors analyzes signals from the sensors to measure quantities of the gasses in breath and present information related to the measured quantities of the gasses in the breath on a display.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1A is a schematic showing an example of a breath analysis device.

FIGS. 1B-1D are schematics showing alternate examples of breath analysis devices.

FIG. 2 is a schematic showing an exemplary system including a breath analysis device, such as the device of FIG. 1A, and a ventilator being used to monitor and support an infant subject.

FIG. 3 is a schematic showing exemplary features that may be included in the devices, systems, and methods for analyzing breaths to detect bacterial infections or other conditions, e.g., using the device shown in FIG. 1A.

FIG. 4A shows an exemplary breath analysis device connected to a tube connected to a nasogastric tube received in a patient to receive breaths from the subject for analysis.

FIG. 4B shows another exemplary breath analysis device connected to a line from a ventilator supporting a subject for receiving breaths from the subject for analysis.

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

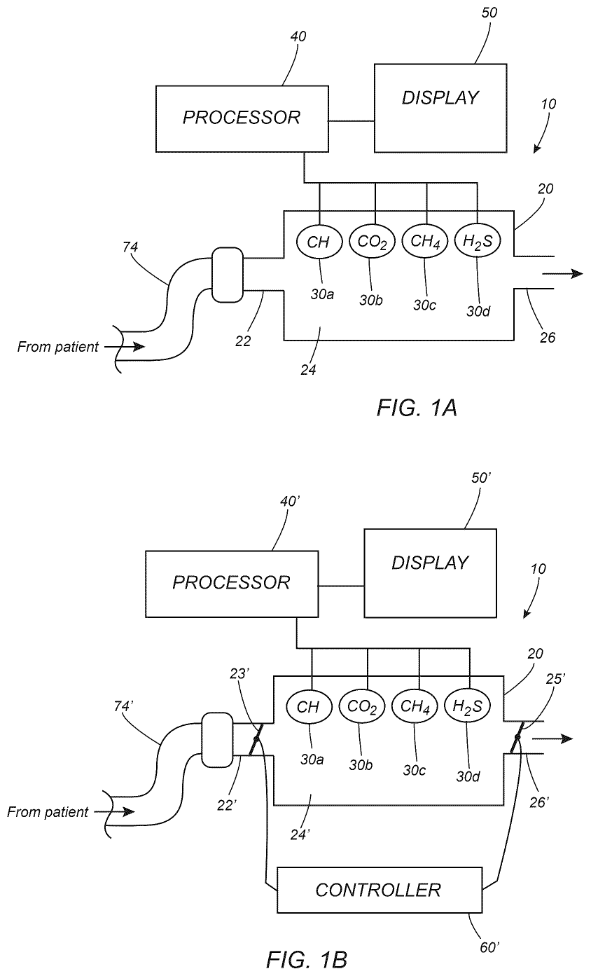

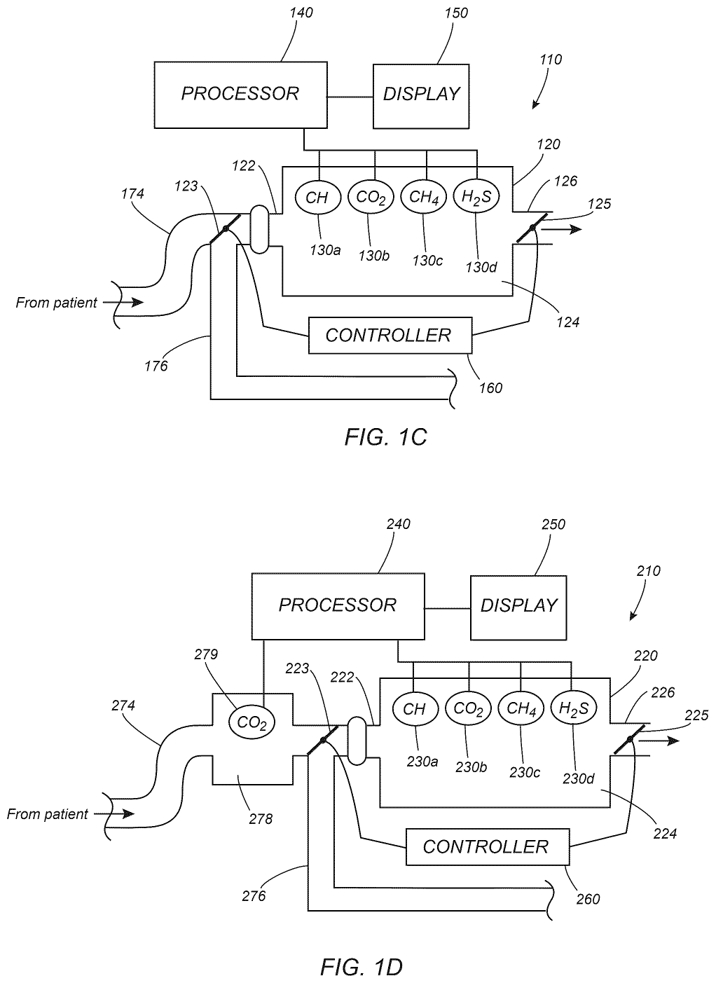

Turning to the drawings, FIG. 1A shows an exemplary device 10 for analyzing one or more breaths of a subject that generally includes a housing 20 for receiving breaths from the subject, e.g., receiving periodic exhalation breaths substantially continuously from the subject, that includes a hydrogen gas sensor 30a, and may, optionally, include one or more additional gas sensors, e.g., gas sensors 30b, 30c, 30d configured to measure gasses such as methane, carbon dioxide, and/or hydrogen sulfide, a processor 40, and a display 50. As described further below, the processor 40 may be coupled to the sensor(s) 30 to detect and/or identify the presence, measure quantities, measure rates of change, and/or measure ratios of gas(ses) in breaths from the subject, and, optionally store and/or analyze sensor outputs in relation to time, and present information related to the gas(ses) on the display 50.

For example, the information presented on the display 50 may facilitate a clinician detecting when the subject is experiencing a bacterial infection and/or other condition, such as necrotizing enterocolitis and/or sepsis. In addition or alternatively, the processor 40 may analyze the information from the sensor(s) to automatically detect such infections and/or present risks or warnings on the display 50, as described further elsewhere herein. Although the devices, systems, and methods are described herein as particularly applicable to detecting bacterial infections in neonates or other infants, such as necrotizing enterocolitis, the device, systems, and methods may also be used to detect infections in adult or other patient populations and/or to detect other conditions.

The sensor(s) 30, processor 40, and display 50 may all be provided on or within the housing 20, e.g., such that the device 10 is self-contained. Alternatively, one or more components of the device 10 may be provided separate from the housing 20. For example, a separate display 50 may be provided that is coupled to the housing 20 by one or more cables (not shown) or that may receive wireless signals from the processor 40 to display information.

Optionally, the device 10 may include one or more additional components, e.g., a battery or other power source, memory for storing instructions and/or data, a clock, a communications interface, e.g., a wireless transceiver for communicating with a remote electronic device, and the like (not shown). In addition or alternatively, one or more connectors, such as a clip, strap, and the like (not shown), may be provided on the housing 20 to connect the device 10 to other equipment objects within a clinical setting, e.g., a ventilator, tubing, bed frame, and the like.

In another alternative, the components of the device 10 may be incorporated directly into other medical equipment, such as a ventilator, monitor, oxygen delivery device, and the like, to provide the additional functionality described elsewhere herein to other equipment.

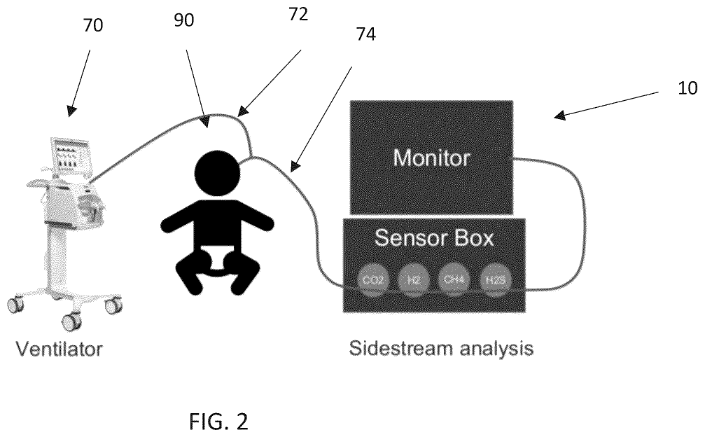

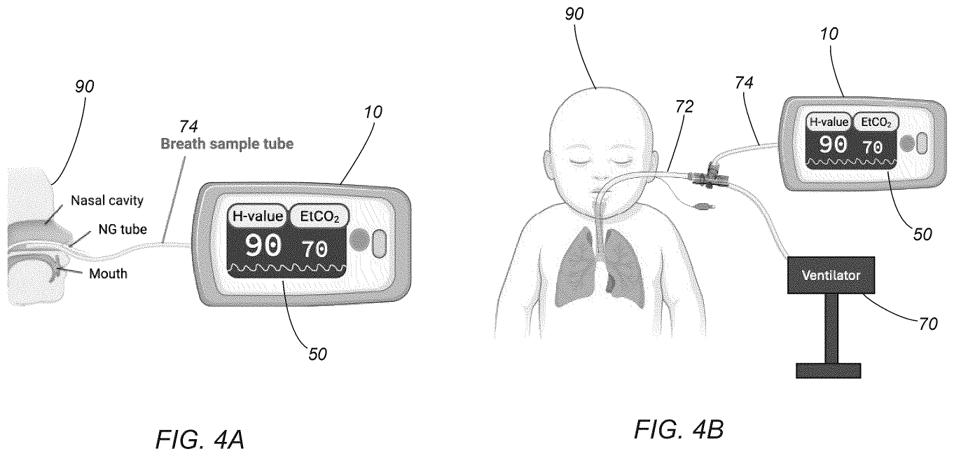

In the example shown in FIG. 1A, the housing 20 includes an inlet 22 communicating with an interior chamber 24 for receiving breath flow such that the sensor(s) 30 are exposed to exhalation breaths from the subject, and an outlet 26 for releasing the breaths after analysis. For example, the inlet 22 may include a port configured to be connected to tubing, e.g., tubing 74 shown in FIG. 2 or 4A, to deliver exhaled air from a subject 90 into the housing 20, as described further elsewhere herein. The outlet 26 may include a port configured to be connected to tubing or may simply be a vent (not shown) to release breath gasses within the housing 20 into the ambient atmosphere around the housing 20 after analysis.

In one example, breaths delivered into the housing 20 from the subject may pass freely through the housing 20, e.g., received into the inlet 22 from tubing 74 delivering exhaled air from a subject 90, e.g., as shown in FIG. 4A, through the chamber 24, and out the outlet 26 using pressure within the tubing 74 from the subject's own breathing or enhanced by pressures generated by a ventilator 70 or other device. Alternatively, a pump or other pressure source (not shown) may be provided within the housing 20 or coupled to the inlet 22 to enhance delivering breaths into the housing 20 and/or removing breath gasses after analysis.

The sensor(s) 30 may be disposed within the chamber 24 such that the sensor(s) 30 are exposed to the breaths introduced into the chamber 24. In one example, the sensor(s) 30 may measure gasses in breaths passing through the chamber 24 in real-time, e.g., as the subject exhales during normal breathing. Thus, multiple breaths of the subject may be monitored and analyzed over time, which, as used herein, may be referred to as being monitored “substantially continuously,” even though sequential exhalation breaths are separated by inhalation breaths.

Alternatively, as shown in FIG. 1B, the housing 20′ may include one or more valves, e.g., an inlet valve 23′ and an outlet valve 27′, for opening and closing the inlet 22′ and/or outlet 26′, respectively. For example, the device 10′ may include a controller 60′ that is coupled to the valves 28′ and/or processor 40′ that is configured to open the inlet valve 23′ to receive individual breaths or samples into the chamber 24′ and then close the valve 23′ to expose the sensor(s) 30 to the gasses. After analyzing the breaths, the controller 60′ may open the outlet valve 27′to release the breaths through the outlet 26′. Optionally, both valves may be opened and closed simultaneously such that pressure at the inlet 22′ may introduce new breath gasses while evacuating the previous gasses out the outlet 26′.

Alternatively, the controller 60′ may open the valves independently to receive and release gasses. In this alternative, the device may include a pump or other pressure source (not shown) to pull breath in through the inlet 22′ and/or evacuate the breath through the outlet 28.′

Alternatively, as shown in FIG. 1C, a breath analysis device 110 may be provided that includes multiple flow paths, in which breaths are directed for sorting and analysis of distinct portions of the breath cycle. For example, a flow path may be provided into the inlet 122 and a secondary flow path 176 may be provided that bypasses the device 110. Directing breaths between the inlet 122 and secondary flow path 176s may be facilitated by one or more directional valves, such as inlet valve 123 and/or outlet valve 125. Optionally, the valve(s) may be opened/closed or otherwise controlled by controller 160 by factors including but not limited to carbon dioxide, pressure, and/or flow rate.

Alternatively, as shown in FIG. 1D, the device may have a pre-chamber 278 for receiving and analyzing breaths prior to sorting distinct portions of the breath cycle into separate flow paths. For example, the pre-chamber 278 may include sensors, such as a carbon dioxide sensor 279 and/or other sensors, such as pressure, temperature, moisture, and/or flow rate sensors, that may analyze properties of the breaths. The flow exiting the pre-chamber 278 may be directed along multiple flow paths, i.e., into inlet 222 or secondary flow path 276, as desired. This bypass may be facilitated by one or more directional valves, such as inlet valve 223 and/or outlet valve 225. The valve(s) may be controlled by the controller 260 using one or more factors determined through measurements in the pre-chamber 278.

Returning to FIG. 1A, the sensor(s) 30 may include one or more sensors to measure gasses, e.g., a hydrogen sensor 30a, and, optionally, may include one or more additional sensors 30b-30, e.g., for measuring one or more of methane, carbon dioxide, and hydrogen sulfide, e.g., to measure the presence of and/or concentration of the gas(ses). For example, as shown, a hydrogen sensor 30a may be provided to identify the presence and/or concentration of hydrogen in exhalation breaths from the subject. Detecting concentrations of hydrogen may alone be sufficient to detect if the subject is experiencing a bacterial infection, such as necrotizing enterocolitis or sepsis. In addition or alternatively, the sensor(s) 30 may include multiple sensors to measure different gasses, e.g., a hydrogen sensor 30a along with one or more of a carbon dioxide sensor 30b, a methane sensor 30c, and a hydrogen sulfide sensor 30d, which may enhance detecting bacterial infections and/or provide additional analysis.

The sensor(s) 30 may be configured to quantify the absolute or relative amounts of the gas(ses) present in breaths and/or the rate of change of the gas(ses), e.g., generating signals that are analyzed by the processor 40 to identify one or more of concentrations of the gas(ses) in parts per million or parts per billion, the relative ratios of gasses, and the concentration of gas(ses) over a period/as a function of time, and/or may simply identify that the amount of gas(ses) present exceeds a predetermined threshold. For example, the sensor(s) 30 may include infrared sensors (including non-dispersive infrared sensors), electrochemical sensors, or sensors using laser absorption spectroscopy to identify and/or measure molecules of the desired gas(ses) present in breath. Other sensors that may be used include chemically-sensitive field-effect transistors (ChemFET), catalytic bead sensors, metal oxide semiconductor (MOS) sensors, gas chromatography (GC) sensors, photoionization detectors (PID), flame ionization detectors (FID), thermal conductivity detectors (TCD), mass spectrometry (MS), photoacoustic spectroscopy (PAS), Raman spectroscopy, chemiluminescence analyzers, Fourier-transform infrared (FTIR) spectroscopy, and/or colorimetric gas detection tubes, and the like.

The sensor(s) 30 may be capable of detecting and/or measuring gas(ses) in real time as exhalation breaths from the subject pass by the sensor(s) 30 within the housing 20, e.g., in the device 10 shown in FIG. 1A as described previously. Alternatively, if the sensor(s) 30 require a minimum period of time to analyze breath gasses, the gasses may be captured within the housing 20 to expose the gasses to the sensor(s) 30 for sufficient time to allow the sensor(s) 30 to measure the gas(ses) and then released after analysis, e.g., using the device 10′ shown in FIG. 1B.

The processor 40 may receive signals from the sensor(s) 30 to analyze the signals to identify the presence, measure quantities, measure rates of change, and/or measure ratios of the gas(ses) within breaths. Information related to the presence and/or concentration of the gas(ses) may then be presented on the display 50, e.g., as numerical values, graphical representations, and the like. Optionally, the processor 40 may analyze the data from the sensor(s) 30, e.g., using an algorithm to analyze trends in the received sensor data, which may be presented on the display 50 to facilitate a clinician identifying changes in the subject's condition. For example, a graph or other time-based representation may be shown on the display 50 to reflect any changes and/or rates of changes in the quantities of the measured gas(ses). Optionally, the algorithm may also incorporate additional factors in data analysis, including the subject's gestational age at birth, current gestational age, chronological age, weight, diet, medications, and/or other factors to enhance monitoring the subject and/or projecting changes in their condition.

The processor 40 may simply present the information on the display 50 such that a clinician may observe the information and assess the subject's condition. For example, the clinician may compare the displayed gas levels to known condition benchmarks to determine whether the subject has an infection, such as necrotizing enterocolitis, and take appropriate action based on the information. Alternatively, the processor 40 may analyze the data from the sensor(s) 30 to automatically identify trends and/or conclude that the subject has an infection, which may be presented on the display 50.

The display 50 may include any visual output device, such as a LCD or other color or black and white screen. Optionally, the display 50 may include additional features, e.g., a user interface to allow a user to operate the device 10. For example, the display 50 may be a touchscreen that includes one or more menus, allowing a user to activate and control the device 10 and/or configure information being presented. Alternatively, the device 10 may include an additional user interface, e.g., one or more buttons, a keypad, keyboard, and the like to allow a user to operate the device 10, and/or modify the information being presented on the display 50.

Optionally, the device 10 may include one or more additional output devices in addition to or instead of the display 50. For example, the device 10 may include a speaker (not shown), which may be coupled to the processor 40 for providing outputs such as status of the device 10, warnings related to changes or trends in the gas(ses) being measured, and the like. For example, the processor 40 may be configured to generate an alarm that provides visual and/or auditory alerts when certain gas thresholds are passed. These thresholds may be based on sensor output values, the results of the data processing analysis, and/or trends of such values and analysis related to the gas(ses) being identified.

Optionally, the device 10 may include one or more features to confirm that breath samples analyzed by the device 10 reflect accurate exhalation breaths from the subject. For example, if the sensor(s) 30 includes a carbon dioxide sensor 30b, the processor 40 may analyze the breath gasses to determine concentration of carbon dioxide in the breath. If the processor 40 confirms that the carbon dioxide levels fall within an acceptable range corresponding to normal exhalation breaths, the processor 40 may include the resulting gas analysis for presentation on the display 50. If the carbon dioxide levels fall outside the acceptable range, the processor 40 may discard the resulting gas analysis and/or may present warnings on the display 50 to notify the clinician. For example, if a mask or nasal cannula is used to collect exhalation breaths from the subject and somehow moves away from the subject's face, e.g., is bumped or falls off, the captured gasses may no longer represent exhalation breaths from the subject but may simply include ambient air, which may lead to false negatives for infection.

Alternatively, FIG. 1D shows an exemplary device 210 in which breaths may be initially analyzed in a pre-analysis chamber 278, e.g., to detect the level of carbon dioxide within the breaths. If the breaths fall within normal ranges, the valve 223 may be positioned to open the path to the inlet 222 and deliver the breath(s) into the device 210 for analysis. If the breath(s) fall outside normal ranges, the valve 223 may be positioned to bypass the device 210 and discard the breath(s) along the secondary flow path 278.

In addition or alternatively, other breath confirmation sensors or mechanisms may be included to confirm that the captured gasses reflect actual exhalation breaths from the subject. For example, the device 10 or 210 may include a moisture or water vapor sensor or a temperature sensor (not shown), and the processor 40 or 240 may analyze signals from the sensor(s) to confirm that the moisture content or temperature of the breath fall within ranges for normal exhalation breaths.

The devices herein may be included in systems for analyzing breath gasses from a subject, such as a neonate or other infant patient, to detect infections, such as necrotizing enterocolitis or sepsis. For example, as shown in FIGS. 2 and 4B, the device 10 may be used in conjunction with a ventilator 70 or other piece of medical equipment to detect and/or monitor a subject 90 for potential infection. As shown, the ventilator 70 includes tubing 72 coupled to a port of the ventilator 70, and side-stream tubing 74 may be connected to the tubing 72 to deliver at least some of the subject's exhalation breaths to the device 10 for analysis, as described elsewhere herein. Alternatively, the device 10 may be positioned in a main-stream position, e.g., directly in line with tubing drawing exhaled air from the subject.

In the example shown, tubing 72 removes exhaled air from the subject 90 and, at least a portion of the exhaled air passing through the tubing 72 is directed through the side-stream tubing 74 into the device 10 for analysis, e.g., as described previously. Alternatively, the tubing 72 may be coupled to other equipment, e.g., an exhaust valve of a capnography device being used to monitor the subject. For example, the exhaust valve may provide sufficient pressure to deliver exhaled air from the subject into the device 10.

In further alternatives, tubing may obtain exhaled air directly from the subject. For example, a face mask may be placed over the subject's face, or a nasal cannula may be inserted into the subject's nose, or a tube may be placed in the subject's nose or mouth to capture exhaled air that is delivered to the device 10 for analysis. Optionally, any of these systems may include a suction mechanism to enhance drawing the subject's breath/exhaled air. Such suction mechanisms may include a fan, pump, motor, or other pressure source that delivers pressure within the tubing to draw exhaled air and deliver it to the device 10 for analysis.

In a further alternative, e.g., as shown in FIG. 4A, if the subject is intubated or has a feeding tube, tubing 74 may be clipped or otherwise connected to such tube(s) to position an inlet of the tubing adjacent to or within the subject's nose or mouth to capture exhaled air, which may be delivered to the device 10 for analysis.

In any of these alternatives, the device 10 may be positioned to receive exhalation breaths from a subject to monitor the subject's condition and/or status of infection, such as necrotizing enterocolitis or sepsis. The sensor(s) may detect the presence and/or measure the concentration of the target gasses in real-time as the breath passes through the device 10, as described elsewhere herein. Alternatively, the sensor(s) may be activated or analyze breaths in periodic intervals, e.g., corresponding to individual breaths, a desired subset of individual breaths, e.g., every second, third, or other interval of breaths, or portions of individual breaths, e.g., the start, middle or end of breaths. For example, the processor 40 may identify when the subject exhales, e.g., due to changes in pressure at the inlet 22 and/or from data received from a ventilator or other equipment monitoring the subject, whereupon the sensor(s) 30 may be activated to measure gas(ses) in breath which correspond to periods of exhalation. Thus, although breaths may be received within the device 10 only intermittently, i.e., from exhalation breaths between inhalation breaths, the analysis by the device 10 would still be considered substantially continuous, since breaths are monitored over an extended period of time, e.g., to monitor and/or detect changes in gas concentrations in the subject.

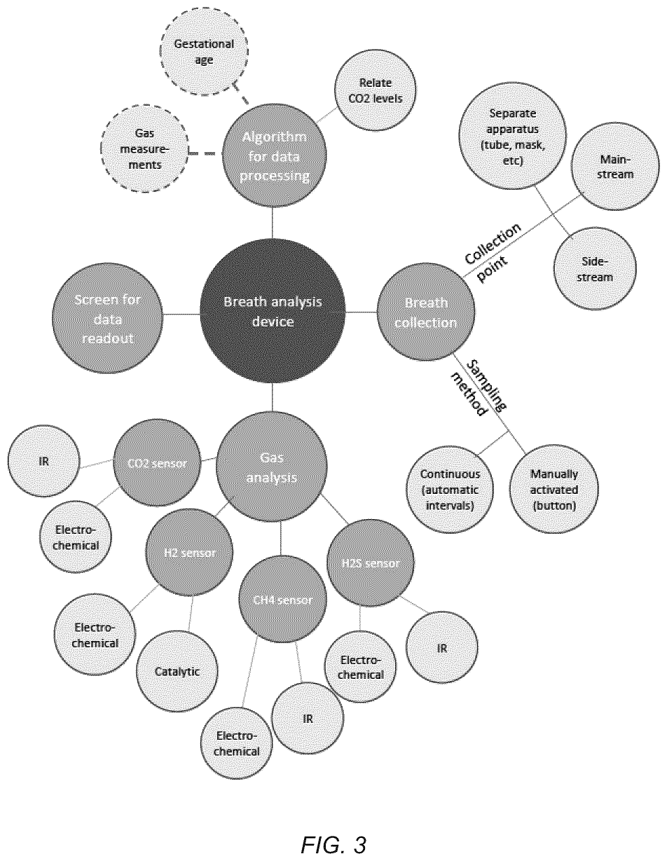

Optionally, as described previously, the processor 40 may also monitor one or more parameters, e.g., carbon dioxide levels, moisture, or water vapor content, and/or temperature in the breath, to confirm that the captured gasses reflect actual exhalation air from the subject. Such monitoring may minimize risk of the measured gasses not reflecting the subject's exhalation air, e.g., if a mask, nasal cannula, tube, or inlet is moved away from the subject's nose or mouth. FIG. 3 shows examples of potential features that may be included in any of the device, methods and systems described herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A device for analyzing breaths of a subject over multiple breath cycles to detect necrotizing enterocolitis or sepsis, comprising:
a housing comprising an inlet for delivering breaths from the subject into the housing;
a hydrogen sensor within the housing;
a display; and
a processor coupled to the hydrogen sensor to analyze signals from the hydrogen sensor to measure hydrogen in the breaths and coupled to the display for presenting information related to the measured quantity of hydrogen in the breaths.

2. The device of claim 1, further comprising:
an apparatus for substantially continuous capture of breaths over a period of multiple breath cycles communicating with the inlet.

3. The device of claim 1, further comprising a carbon dioxide sensor within the housing and wherein the processor is coupled to the carbon dioxide sensor to analyze signals from the carbon dioxide sensor to measure carbon dioxide in the breaths and present information related to the measured quantity of carbon dioxide on the display.

4-6. (canceled)

7. The device of claim 1, wherein the processor is configured to monitor breaths from the subject delivered via the inlet and present information related to measured quantities as a function of time.

8. The device of claim 1, wherein the housing comprises an outlet for removing the breaths from the housing after analyzing the breaths.

9. The device of claim 8, wherein the housing comprises one or more chambers communicating with the inlet and outlet such that the breaths pass continuously through the chamber from the inlet to the outlet for analysis.

10. The device of claim 8, wherein the housing comprises one or more chambers communicating with the inlet for receiving the breaths and a controller for selectively closing the chamber to isolate the chamber after receiving an individual breath, or portion of the breath, from the inlet and open the chamber to release the individual breath after analysis through the outlet.

11. The device of claim 8, wherein the outlet is configured to release the breaths into an ambient environment outside the housing.

12. The device of claim 1, wherein the display is mounted on the housing or is separate from the housing.

13. (canceled)

14. The device of claim 1, further comprising a breath confirmation sensor, the processor configured to analyze signals from the breath confirmation sensor to confirm that the breaths received within the housing correspond to actual breaths from the subject.

15. The device of claim 14, wherein the breath confirmation sensor comprises a carbon dioxide sensor exposed to the breaths, the processor configured to analyze the signals to confirm that the carbon dioxide levels in the breaths correspond to normal exhalation from the subject before analyzing the measured quantity of hydrogen in the breaths to detect one or more conditions of the subject.

16. The device of claim 14, wherein the breath confirmation sensor comprises a moisture or water vapor sensor exposed to the breaths, the processor configured to analyze the signals to confirm that the moisture or water vapor levels in the breaths correspond to normal exhalation from the subject before analyzing the measured quantity of hydrogen in the breaths to detect one or more conditions of the subject.

17. The device of claim 14, wherein the breath confirmation sensor comprises a temperature sensor exposed to the breaths, the processor configured to analyze the signals to confirm that the temperature levels in the breaths correspond to normal exhalation from the subject before analyzing the measured quantity of hydrogen in the breath to detect one or more conditions of the subject.

18. (canceled)

19. The device of claim 1, further comprising a clock or processor which stores, analyzes, and/or presents information in relation to time.

20. The device of claim 1, comprising a connector for attaching the housing to one of tubing and a piece of equipment.

21. A system for analyzing breaths from a subject to detect infection, comprising:
a device for analyzing breaths of a subject comprising a housing comprising an inlet for delivering multiple breath cycles from a subject into the housing; a hydrogen gas sensor within the housing; a display; and a processor coupled to the sensors to analyze signals from the hydrogen sensor to measure the quantity of the gas in the breath and coupled to the display for presenting information related to the measured quantities of the gasses in the breath;
tubing comprising a first end coupled to the inlet; and
medical equipment configured to receive exhalation breaths from the subject and coupled to a second end of the tubing to deliver breath from the subject to the device.

22-28. (canceled)

29. The system of claim 21, further comprising one or more additional gas sensors within the housing configured to measure gasses including one or more of methane, carbon dioxide, and/or hydrogen sulfide.

30. The system of claim 29, wherein the processor is coupled to the one or more additional gas sensors to analyze signals from the one or more additional sensors to measure quantities of the gasses in the breath and coupled to the display for presenting information related to the measured quantities of the gasses in the breath.

31. A method for analyzing breath from a subject to detect infection, comprising:
delivering multiple breath cycles from the subject into a breath analysis device to expose the breath to one or more gas sensors which include a hydrogen sensor, whereupon a processor coupled to the sensors analyzes signals from the sensor to measure quantities of the gasses in the breath and presents information related to the measured quantities of the gasses in the breath on a display.

32-49. (canceled)
